(19) United States
(12) Patent Application Publication
(10) Pub. No.: US 2024/0252704 A1
(43) Pub. Date: Aug. 1, 2024

(54) Portable Self-Controlled UVC Disinfection Device With Smart Sensors

(71) Applicant: Gerald Ho Kim, Spokane, WA (US)

(72) Inventor: Gerald Ho Kim, Spokane, WA (US)

(21) Appl. No.: 18/104,464

(22) Filed: Feb. 1, 2023

(51) Int. Cl. A61L 2/24; A61L 2/10

(52) U.S. Cl. CPC A61L 2/24; A61L 2/10; A61L 2202/11; A61L 2202/14; A61L 2202/16

(57) ABSTRACT

A portable ultraviolet-C (UVC) disinfection device includes a printed circuit board (PCB) module, a heatsink, a fan, a switch, a control circuit, a display screen, and a battery. The PCB board module includes a housing, a UVC array module, a UVC power sensor, a distance measurement sensor, a collimating lens and a PCB that are mounted on the housing and configured to emit a UVC light on a target surface to disinfect the target surface with a disinfection UVC dosage based on a calculated exposure time.

### BACKGROUND

A portable ultraviolet-C (UVC) light-emitting diode (LED) disinfection has been developed to disinfect on a contaminated surface. One of limiting usage of the UVC disinfection device is to determine the effectiveness of disinfecting all virus and bacteria on the surface of disinfecting object. Currently it is difficult to monitor the killing rate of such virus or bacteria; therefore, the portable UVC disinfection device is needed to determining the effective killing rate based on smart feedback sensor system and a real-time adjusting of UVC exposure dosage for a reaching targeted disinfection rate of more than 99.9% kill factor.

Due the difficulty of real-time monitoring the spreading of germs on a contaminated surface, the portable UVC disinfection device has to operate by predetermining an exposure dosage and time to achieve the kill rate of 99.9% or better. The portable UVC disinfection device has to calculate the light intensity impinging on the disinfecting surface. The light intensity is calculated by the distance from the disinfecting surface and the UVC power of the UVC light source that has a predetermined illumination pattern designed by a combination of lenses that collimate the UVC light. The UVC light generated by the UVC array module is difficult to collimate due to the multi-array configuration and the UVC light needs to maintain less than 60 degrees of diverging angle in order to get a relatively high UVC light intensity. At present time the UVC array has to more than 3 by 3 array size to deliver a relatively high UVC light intensity. The UVC array module can be built by 4 by 4 or 5 by 5 configuration for higher light intensity. It will be better to have a high UVC light intensity in order to deliver a high UVC light intensity for disinfecting the contaminated surface quickly. The light intensity is measured by a distance sensor and the illumination pattern projected on the disinfecting surface to calculate UVC light power per unit area, normally a unit of milliwatt/centimeter. The distance can be measured by a time-of-flight sensor or proximity sensor. The range of distance measurement can be in a 0.1 to 5 meters depending on the UVC light intensity and a tight focus UVC light by the collimating lenes.

For example, the UVC LED emits 1 watt of UVC light in 20 degrees of illumination pattern in 1 meter distance. The UVC LED is projecting 35.2 cm diameter area with a project area of 977 cm². Even though the light intensity is not uniform, but 1 watt of UVC light generates about 1 mW/cm² of light intensity. Typical virus needs about 10 mJ of dosage to reach a 99.9% of kill factor and the UVC disinfection device has to expose for at least 10 seconds [1 mW/cm²=1 mJ/(second×cm²)]. The UVC disinfection device has to hold for 10 seconds in same area to kill 99.9% of virus and the bacteria requires higher dosage. In this example the UVC disinfection device has to maintain the exposure distance of 1 meter during the disinfection time of 10 seconds and the kill factor can be reduced or increased depending on maintaining the exposure distance. During the disinfection time it can be difficult to maintain the fixed distance and this invention adds a real-time function of adjusting the disinfection time depending on the exposure distance measurement in real-time based. For instance, the UVC disinfection device is moved out from the 1-meter distance to 1.5-meter distance by the distance measurement sensor and the exposure time will be quickly calculated to increase the exposure time based of the light intensity stored in data base. It is difficult to measure real-time the exposure area by a UVC camera sensor and the exposure area by the distance information is calculated based on the illuminator lens design at the initial design.

The invention contains a method of self-correcting the UVC illumination dosage based on a fixed optical illumination pattern to deliver an enough dosage for reaching the targeted kill factor of germs. The UVC disinfection device consists with a high power UVC LED array, distance measurement sensor and UVC photodiode. The UVC power is constantly measured by the UVC photodiode along with the distance between the UVC LED array and the UVC exposed area. This information is used to calculate the exposure time to disinfect the predetermined kill dosage of germs. In order to calculate the accurate dosage of a certain kind of virus or bacteria the killing time of the virus or bacteria is stored in the data base in the device and the projected UVC light pattern is also stored in the device. For example, the UVC LED light propagates as a 120-degree diverging angle of cone shape propagation pattern without any optics or reflectors and it projects a flat-top light pattern on the disinfecting surface. The portable UVC disinfection device can calculate the exposure time based on the UVC light power, object distance and light pattern to calculate the UVC dosage to disinfect the illuminator area.

The most of UVC disinfection device has an issue of determining whether all germs are safely disinfected by the UVC disinfection device. The UVC disinfection device is depended on a fixed amount of time that is instructed by the device manufacture for disinfecting all contaminated surface area. It is difficult to know whether enough UVC dosage is delivered at a given UVC light intensity. Especially a mercury UVC lamp and uncollimated UVC LED light can be very difficult to determine the UVC dosage that is deliver to the contaminated surface. However, this invention eliminates an uncertainty of guessing the exposure time to kill all germs by device user. The portable UVC disinfection device is designed to deliver a correct amount of UVC dosage with any interference with the device user and the device automatically increase and decrease a UVC exposure time if the user moves around the device while the device is disinfecting the contaminated surface. The portable UVC disinfecting device allows to set a certain virus or bacteria to disinfect in 99.9% and the UVC power and object distance is continuously measured and calculated a correct exposure time to reach the targeted kill factor of 99.9% or better.

The UVC light is invisible to our eyes and it is difficult to cover all contaminated surface area. In order to assist on the effectiveness and well coverage of UVC light exposure a few color LED chips are integrated in the UVC color array module. For example, three color LEDs such as red, green and blue are integrated in 3 by 3 UVC LEDs array making a 3 by 4 array module. Each color LEDs are integrated in the UVC color array module for overlap with UVC light pattern to provide a visual effect of the disinfecting area. While the UVC light are turned on or off, the color LEDs are independently turned on or off. For instance, a green color LED can be illuminated an area before the UVC array is turned on and a user can aim correctly to the UVC disinfecting area. Then, the UVC light in the UVC color array module is turned on and a blue color LED is turned on during the UVC light is turned on. Finally, a red color LED is turned on for indicating the turning off of the UVC light. The multi-color display allows assisting a proper disinfection and safety of the device user.

The potable and self-controlled UVC disinfection device can be used for killing all germs in all public area such as hospital, hotel, school, airline, restaurant, airport and many more. The color assisted UVC disinfection device can help to deliver highly effective disinfecting a contaminated area and protecting from UVC light exposure. This portable UVC disinfection device can be easily carried and operated by user for disinfecting any germs.

### BRIEF DESCRIPTION OF THE DRAWINGS

FIG. 1 is a functional diagram of a UVC illumination device under a proposed scheme in accordance with the present disclosure.

FIG. 2 is a flow chart showing an operational process of the UVC illumination device under a proposed scheme in accordance with the present disclosure.

FIG. 3 shows a normal operational process described in FIG. 2 assuming that the UVC disinfection device is standing still in position to maintain the target distance during the duration of disinfection exposure time.

FIG. 4 shows an example of a UVC array module in a 3×3 array arrangement of UVC LEDs under a proposed scheme in accordance with the present disclosure.

FIG. 5 shows a printed circuit board (PCB) module of a UVC illumination device under a proposed scheme in accordance with the present disclosure.

FIG. 6 shows a portable UVC disinfection device under a proposed scheme in accordance with the present disclosure.

FIG. 7 shows a UVC array module of FIG. 4 combined with a red LED, a green LED and a blue LED under a proposed scheme in accordance with the present disclosure.

FIG. 8 shows a PCB module mounted with a new UVC array module that contains all UVC dies and sensors shown in FIG. 5 under a proposed scheme in accordance with the present disclosure.

FIG. 9 shows a portable UVC disinfection device with a PCB module mounted in the portable UVC disinfection device under a proposed scheme in accordance with the present disclosure.

### DESCRIPTION OF PREFERRED EMBODIMENTS

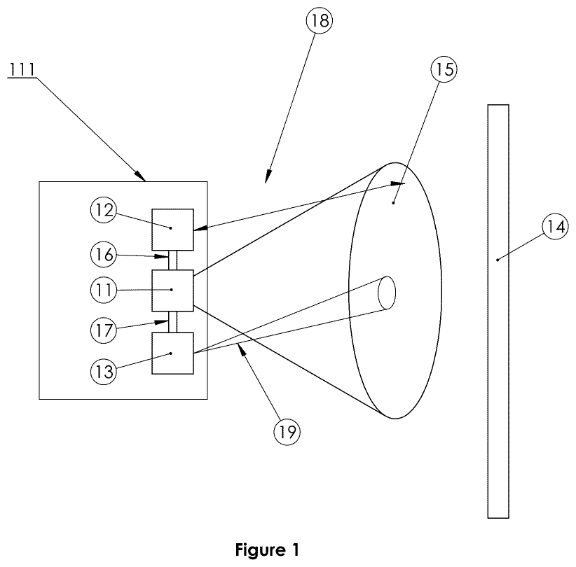

### FIG. 1

FIG. 1 shows a functional diagram of the UVC illumination device (111) is layout with all sensors including a UVC light module (11), distance sensor (12) and UVC power sensor (13) that are connected by wires (16, 17). The UVC light (15) is projected a target (14) and the target distance (18) to the target (14) is shown in FIG. 1. The UVC power sensor (13) of the field-of-view (FOV) (19) is also drawn on this diagram. These sensors are the main components to correctly monitor a proper UVC dosage to deliver on the surface of the target (14) to disinfect a high kill rate (>99.9%) of any germs.

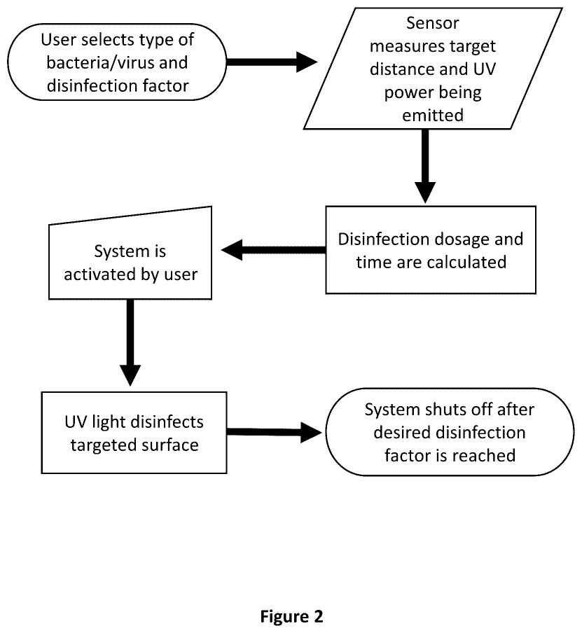

### FIG. 2

The flow chart shows an operational process of the UVC illumination device (111) where a user selects a type of bacteria or virus along with disinfection factor such as 90%, 99%, 99.9% or more. This information has to enter by the user since each bacterium and virus has different disinfection time so that the device can calculate the exposure UVC dosage based on UVC light intensity, UVC illumination pattern and the target distance (18). Both sensors of the distance sensor (12) and UVC power sensor (13) can calculate the exposure time to deliver a correct UVC dosage. After the exposure time is calculated, the user is turned on the UVC illumination device (111) for delivering enough UVC dosage.

This flow chart shows the sequential process of disinfecting the target surface.

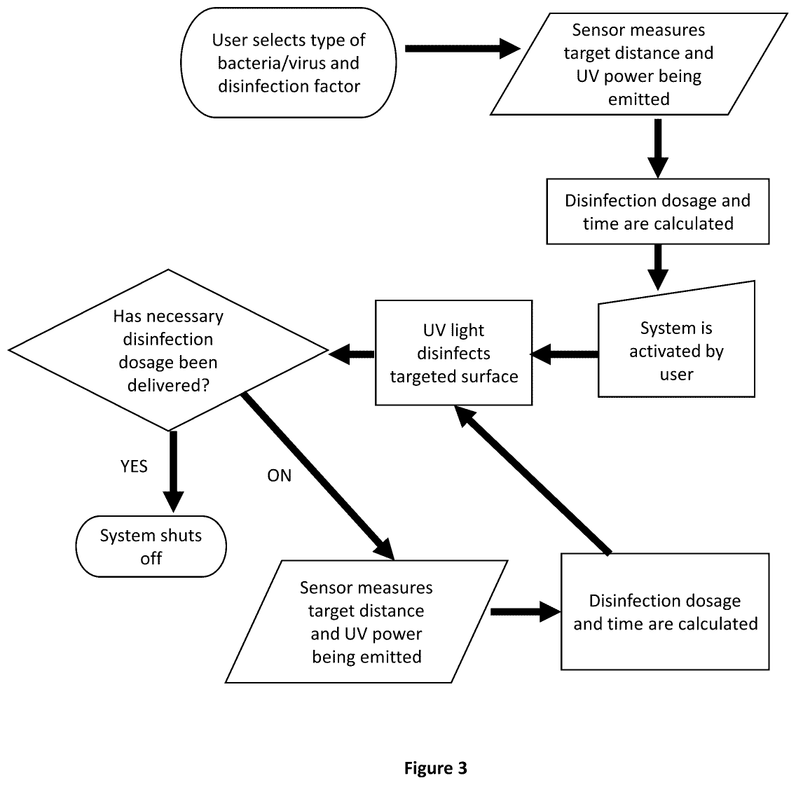

### FIG. 3

FIG. 3 shows a normal operational process described in FIG. 2 assuming that the UVC disinfection device (111) is standing still in position to maintain the target distance (18) during the duration of disinfection exposure time. If the target distance (18) is changed during the exposure time, the UVC dosage may not reach the kill factor for the germs. The UVC illumination device (111) has to recalculate the exposure time to reach kill factor depending on the new target distance. The operational process frequently acquires the target distance (13) and compare to the UVC light dosage based on changing target distance. If the UVC dosage is not reach the kill factor, the UVC exposure time will be added or subtracted from the initial exposure time to reach the targeted UVC dosage. This invention allows any guessing work to deliver a correct UVC dosage to kill all virus and bacteria. Current UVC disinfection device has a fixed timer to turn on the UVC disinfection device without accurately delivering a correct amount of UVC dosage for disinfecting targeted surface.

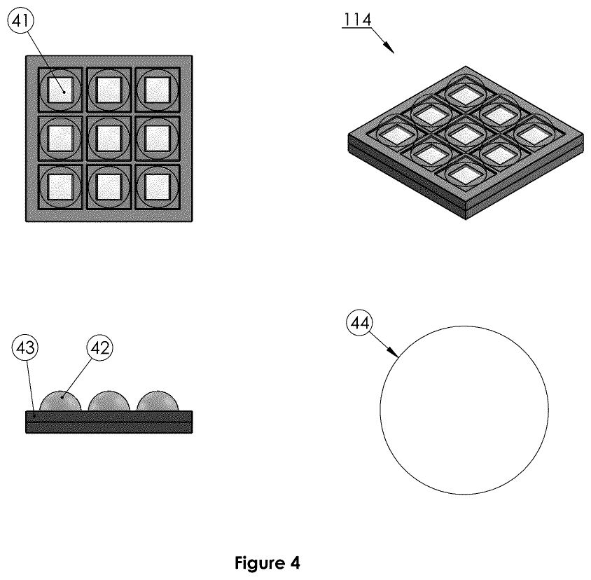

### FIG. 4

FIG. 4 shows an example of the UVC array module (114) in a 3×3 array arrangement of UVC LEDs. UVC dies (41) are placed in 3×3 array package (43) and a small lens (42) is mounted on each UVC dies (41). The small lenses (42) are mounted on the 3×3 array package (43). The UVC illumination pattern (44) is also projected by all UVC light combined by all UVC dies (41). Due to the small lenses (41) mounted on the UVC dies (41) will projected as a slowly diverging UVC light for the device. The UVC array module (114) is same component that is described in the UVC light module (11) in FIG. 1.

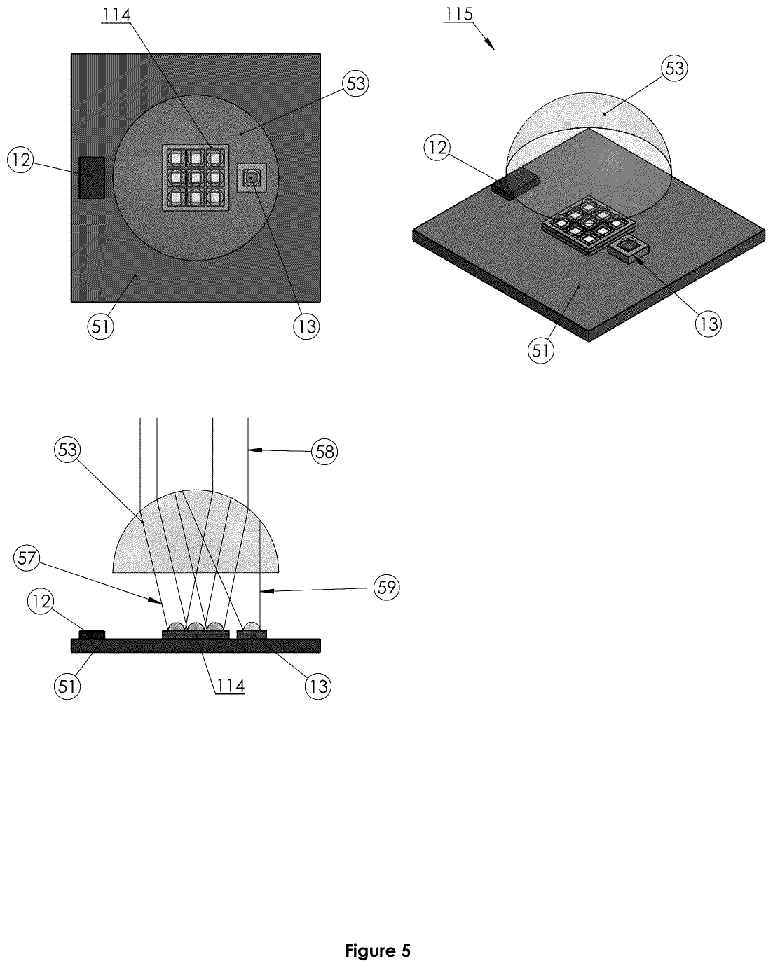

### FIG. 5

FIG. 5 shows a printed circuit board (PCB) module (115) mounted with the UVC array module (114), the UVC power sensor (13), the distance sensor (12), a collimating lens (53) and PCB (51). All these components are integrated on the PCB (51). The UVC power sensor (13) is closely mounted to the UVC array module under the collimating lens (53) to monitor the constant power measurement for the UVC array module (114). The UVC light (57) from the UVC dies (41) through the small lenses (42) enters into the collimating lens (53) to the UVC ray (58) to the target (14). The field-of-view (19) is illustrated as a returning ray (59) into the UVC power sensor (13) through the collimating lens (53).

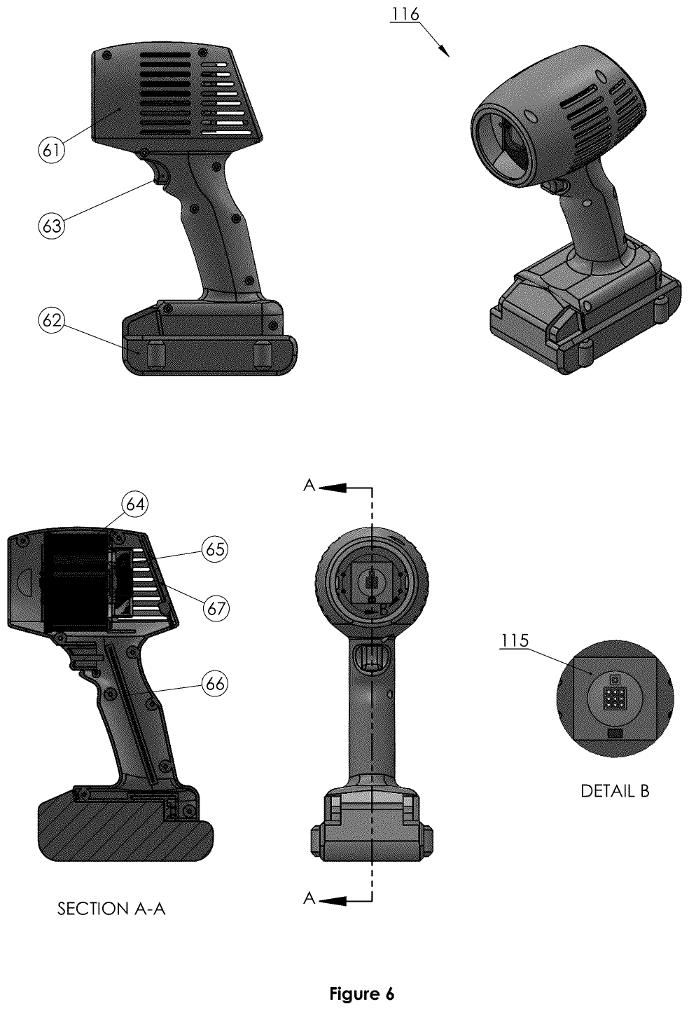

FIG. 6

A portable UVC disinfection device (116) is shown in FIG. 6 and the PCB module (115) is mounted in the portable UVC disinfection device (116). The PCB module (115) is mounted on a heatsink (64) and fan (65) and the PCB module (115) is wired with a control circuit (66) and a screen 67) where the status of the portable UVC disinfection device is displayed. All these components are closed with a housing (61). The portable UVC disinfection device (116) has a switch (63) to turn on/off the PCB module (115) and the portable UVC disinfection device (116) is powered by a rechargeable battery pack (62).

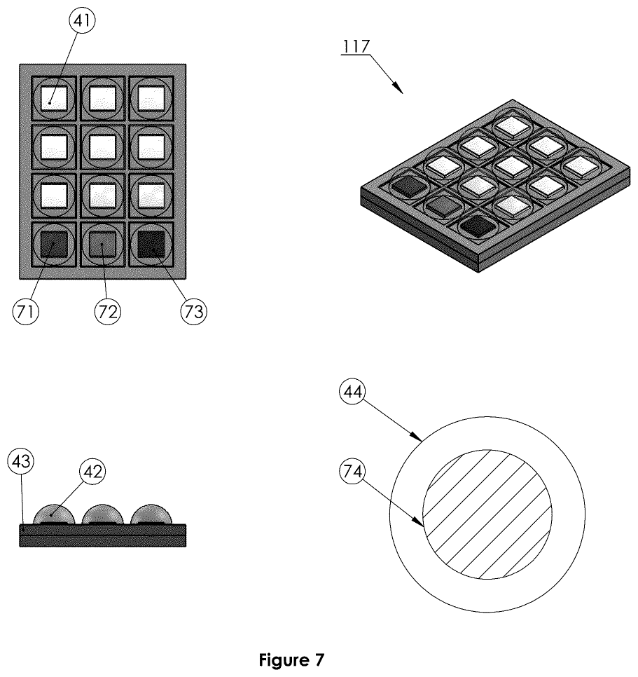

### FIG. 7

The UVC array module (114) in FIG. 4 is combined with a red LED (71), green LED (72) and blue LED (73). Nine UVC dies (41) are integrated in the UVC array module (117) along with all color LEDs. These color LEDs can turn on and off for a status of the UVC dies (41), disinfection time of the portable UVC disinfection device (116), and all other status mode such as low power UVC LED. The UVC illumination pattern (44) is overlapped with an illumination pattern of all color LEDs for marking on the disinfection area of the target (14).

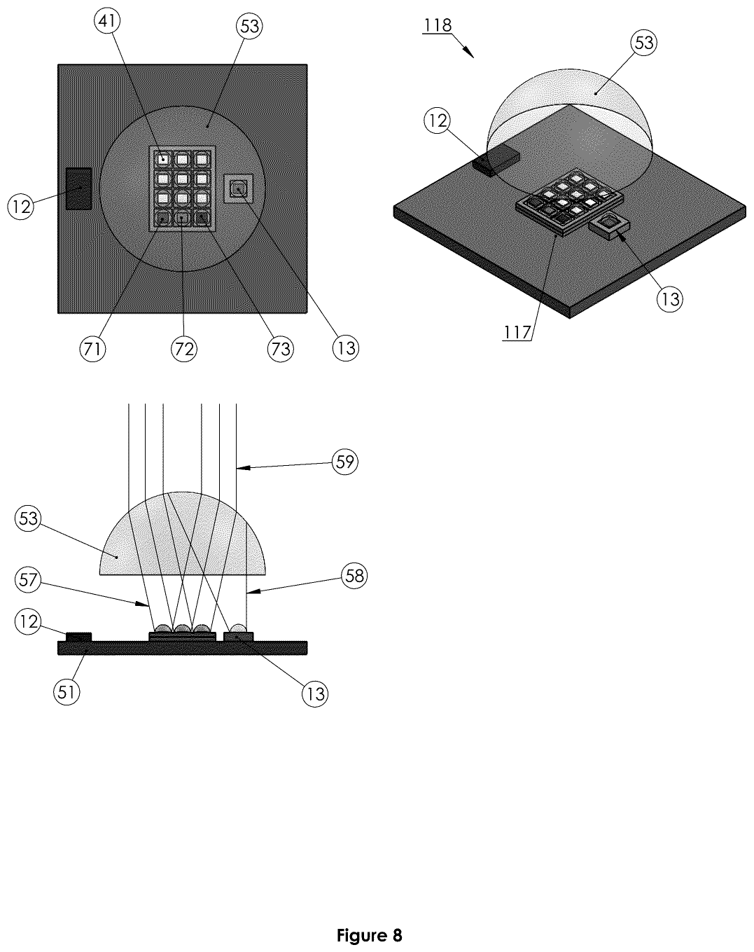

### FIG. 8

A PCB module (118) is mounted with the new UVC array module (117) that contains all UVC dies (41) and sensors shown in FIG. 5. FIG. 8 shows a PCB module (118) mounted with the UVC array module (117), the UVC power sensor (13), the distance sensor (12), a collimating lens (53) and PCB (51). All these components are integrated on the PCB (51). The UVC power sensor (13) is closely mounted to the UVC array module under the collimating lens (53) to monitor the constant power measurement for the UVC array module (117). The UVC light (57) from the UVC dies (41) through the small lenses (42) enters into the collimating lens (53) to the UVC ray (58) to the target (14). The field-of-view (19) is illustrated as a returning ray (59) into the UVC power sensor (13) through the collimating lens (53).

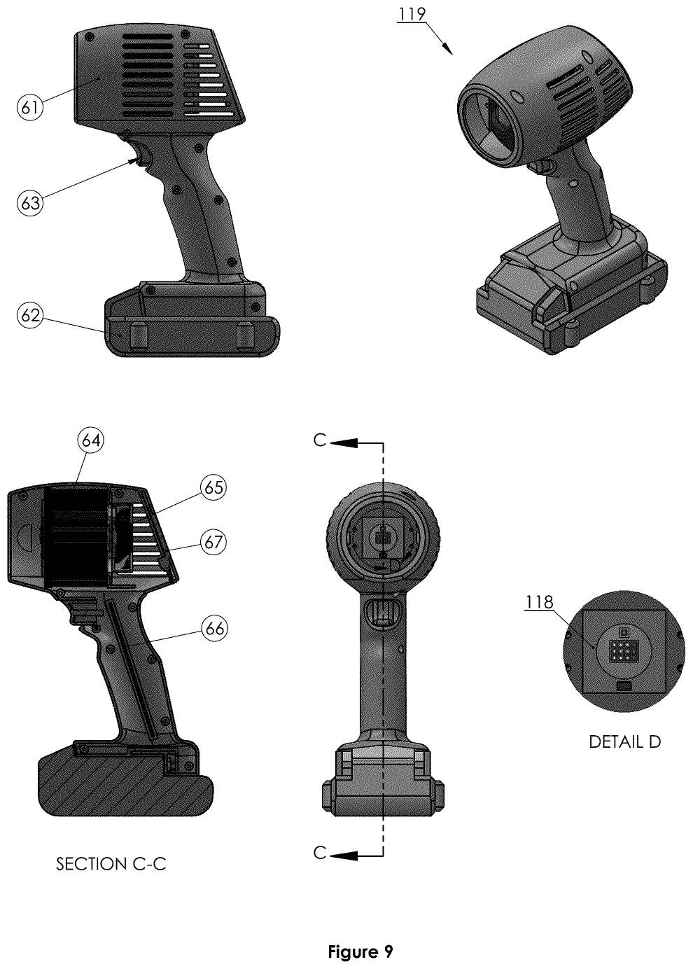

### FIG. 9

A portable UVC disinfection device (119) is shown in FIG. 9 and the PCB module (118) is mounted in the portable UVC disinfection device (119). The PCB module (118) is mounted on a heatsink (64) and fan (65) and the PCB module (118) is wired with a control circuit (66) and a screen 67) where the status of the portable UVC disinfection device is displayed. All these components are closed with a plastic shell (61). The portable UVC disinfection device (119) has a switch (63) to turn on/off the PCB module (118) and the portable UVC disinfection device (119) is powered by a rechargeable battery pack (62).

What is claimed:

1. A portable ultraviolet-C (UVC) disinfection device, comprising:
a printed circuit board (PCB) module;
a heatsink;
a fan;
a switch;
a control circuit;
a display screen; and
a battery,
wherein the PCB module comprises a housing, a UVC array module, a UVC power sensor, a distance measurement sensor, a collimating lens and a PCB that are mounted on the housing and configured to emit a UVC light on a target surface to disinfect the target surface with a disinfection UVC dosage based on a calculated exposure time.

2. The portable UVC disinfection device of claim 1, wherein the collimating lens is configured to collimate the UVC light in less than 60 degrees of a full angle.

3. The portable UVC disinfection device of claim 1, wherein the distance measurement sensor comprises a time-of-flight sensor.

4. The portable UVC disinfection device of claim 1, wherein the distance measurement sensor comprises a proximity sensor.

5. The portable UVC disinfection device of claim 1, wherein the UVC array module is configured to produce more than 0.5 watt per centimeter square.

6. The portable UVC disinfection device of claim 1, wherein the UVC power sensor is adjacent to the UVC array module and configured to monitor a portion of the UVC light as a return light from the collimation lens.

7. The portable UVC disinfection device of claim 1, wherein the exposure time is calculated by a distance from the target surface, a UVC light intensity measured by the UVC power sensor, and a UVC illumination pattern so as to achieve a target disinfection level.

8. The exposure time of claim 8, wherein the exposure time is automatically controlled by the control circuit without any interference by a device user.

9. The portable UVC disinfection device of claim 1, wherein the exposure time is automatically increased or decreased according to a change in the disinfection distance so as to achieve a targeted exposure time.

10. A portable ultraviolet-C (UVC) disinfection device, comprising:
a printed circuit board (PCB) module;
a heatsink;
a fan;
a switch;
a control circuit;
a display screen; and
a battery,
wherein the PCB module comprises a housing, a UVC color array module, a UVC power sensor, a distance measurement sensor, a collimating lens and a PCB mounted on the housing that are configured to emit a UVC light on a target surface of an object to cause disinfection by a disinfection UVC dosage for an exposure time.

11. The portable UVC disinfection device of claim 10, wherein the collimating lens is configured to collimate the UVC light in less than 60 degrees of a full angle.

12. The portable UVC disinfection device of claim 10, wherein the distance measurement sensor comprises a time-of-flight sensor.

13. The portable UVC disinfection device of claim 10, wherein the distance measurement sensor comprises a proximity sensor.

14. The portable UVC disinfection device of claim 10, wherein the UVC color array module comprises a compact UVC and color LED dies mounted in more than 3×4 array form.

15. The UVC color array module of claim 14, further comprising at least three color LEDs configured to indicate a status of the UVC LED array.

16. The UVC color array module of claim 15, wherein the UVC light propagates in an emission pattern similar to that of the color LEDs.

17. The portable UVC disinfection device of claim 10, wherein the UVC power sensor is adjacent to the UVC array module and configured to monitor a portion of the UVC light as a return light from the collimation lens.

18. The portable UVC disinfection device of claim 10, wherein the exposure time is calculated by a distance from the target surface, a UVC light intensity measured by the UVC power sensor, and a UVC illumination pattern so as to achieve a target disinfection level.

19. The exposure time of claim 18, wherein the exposure time is automatically controlled by the control circuit without any interference by a device user.

20. The portable UVC disinfection device of claim 10, wherein the exposure time is automatically increased or decreased according to a change in the disinfection distance before reaching a targeted exposure time.
